(19) United States
(12) Patent Application Publication
(10) Pub. No.: US 2025/0073430 A1
(43) Pub. Date: Mar. 6, 2025

(54) MEDICAL DEVICE WITH ADJUSTMENT KNOB AND FEEDBACK MECHANISM

(71) Applicant: Biosense Webster (Israel) Ltd., Yokneam (IL)

(72) Inventor: Michael BANANDO, Glendora, CA (US)

(73) Assignee: Biosense Webster (Israel) Ltd., Yokneam (IL)

(21) Appl. No.: 18/726,336

(22) PCT Filed: Jan. 30, 2023
(86) PCT No.: PCT/IB2023/050780

§ 371 Date: Jul. 2, 2024

Related U.S. Application Data
(60) Provisional application No. 63/305,757, filed on Feb. 2, 2022.

(51) Int. Cl. A61M 25/10

(52) U.S. Cl. CPC A61M 25/10184; A61M 2205/3327; A61M 2205/3334; A61M 2205/502

(57) ABSTRACT

A medical instrument may include an expandable balloon on a distal end and a handle with a knob thereon. The knob may be movable along the handle, and the handle may include a switch disposed between the handle and the knob so that the movement of the knob relative to the handle activates and deactivates a circuit connected to the switch to provide an indication of the position of the knob in relation to the handle to a console. A catheter may include an elongated catheter shaft, an expandable member distal of the elongated catheter shaft, and a handle with a knob disposed thereon. The catheter may include a switch disposed between the knob and handle so that movement of the knob activates and deactivates a circuit connected to the switch.

### CROSS REFERENCE TO RELATED APPLICATIONS

This application claims the benefit of priority under to Paris Convention to prior filed U.S. Provisional Patent Application No. 63/305,757, filed on Feb. 2, 2022, the entire contents of which is hereby incorporated by reference as if set forth in full herein.

### BACKGROUND

Medical catheters may be used in various medical procedures, including cardiac electrophysiologic (EP) mapping and cardiac ablation. Various medical catheters may be employed for cardiac mapping and ablation purposes, including balloon catheters and basket assemblies having expandable members. After ablation is performed, but prior to withdrawal of the medical catheter, irrigation fluid flow may be reduced in rate from a first flow rate to a second flow rate and the medical catheter may be collapsed from an expanded state into a collapsed state.

### SUMMARY

Medical instruments, catheters, and methods of using thereof are disclosed herein. A medical instrument may include an expandable balloon on a distal end and a handle with a knob thereon. The knob may be movable along the handle, and the handle may include a switch disposed between the handle and the knob so that the movement of the knob relative to the handle activates and deactivates a circuit connected to the switch to provide an indication of the position of the knob in relation to the handle to a console. A catheter may include an elongated catheter shaft, an expandable member distal of the elongated catheter shaft, and a handle with a knob disposed thereon. The catheter may include a switch disposed between the knob and handle so that movement of the knob activates and deactivates a circuit connected to the switch.

An aspect of the present invention that is described herein provides a medical instrument including an expandable balloon disposed on a distal end of the medical instrument, a handle disposed approximate a proximal end of the medical instrument, a knob extending from the handle, and a switch disposed between the handle and the knob. The expandable balloon may include an internal volume that is configured to receive inflation fluid. The knob may be movable in relation to the handle along a longitudinal axis of the medical instrument such that when the handle moves in a first direction along the longitudinal axis the expandable balloon truncates in length along the longitudinal axis to allow inflation of the expandable balloon and when the handle moves in a second direction along the longitudinal axis the expandable balloon elongates to facilitate deflation of the expandable balloon. The switch may be disposed between the handle and the knob so that movement of the knob relative to the handle activates and deactivates a circuit connected to the switch and provides an indication of a position of the knob, in relation to the handle, along the longitudinal axis.

According to some embodiments, the switch can be at least one of a proximity sensor switch, an optical sensing switch, a micro level switch, or combinations thereof. In some embodiments, the switch can be further configured to provide one or more of an audible indication, a tactile indication, and a visual indication of the position of the knob in relation to the handle. In some embodiments, the switch can be further configured to provide a signal to a console that is in electrical communication with the switch. The console may indicate a position of the knob in relation to the handle.

In some embodiments, the medical instrument can further include an irrigation pump. The irrigation pump can be in electrical communication with the switch, and the irrigation pump can be configured to switch from a first flow rate to a second flow rate in response to an indication from the switch that the knob has been moved in the second direction to deflate the expandable balloon. The irrigation pump can be configured to provide the inflation fluid to the expandable balloon in response to the knob being moved in the first direction along the longitudinal axis.

In some embodiments, the console may further include a display screen that is in electrical communication with the switch. The display screen can be configured to provide a countdown timer in response to the irrigation pump switching from first flow rate to the second flow rate. In some embodiments, the display screen can be further configured to provide a visual indication of a current flow rate of the irrigation pump selected from the first flow rate and the second flow rate. The first flow rate can be a high flow rate and the second flow rate can be a low flow rate.

According to some embodiments, the display screen can be further configured to provide a visual indication reminding an operator of the medical instrument to verify that the medical instrument is not deflected or torqued before removing the medical instrument from a patient's vasculature.

According to some embodiments, the expandable balloon can include one or more electrodes coupled to an outer surface of the expandable balloon.

According to some embodiments, the medical instrument can be applied in a medical procedure selected from electrophysiology, ablation, sinuplasty, surgery, endoscopy, angioplasty, otolaryngology, and neurology.

In another aspect, a catheter is provided. The catheter may include an elongated catheter shaft having a first lumen and defining a longitudinal axis. The elongated catheter shaft may have a proximal end and a distal end, with the proximal end defining a handle. At a distal end of elongated catheter shaft the catheter may include an expandable member. The expandable member may have a distal end and a proximal end, and an actuator disposed along the longitudinal axis and connected to the distal end of the expandable member to facilitate the movement of the expandable member. The catheter may include irrigation tubing that may extend through the first lumen of the elongated catheter shaft. The irrigation tubing may have a second lumen, and a distal end of the irrigation tubing may terminate at the proximal end of the expandable member. The catheter may include a knob, which is fitted on the handle. The knob may be moveable along the longitudinal axis to control the actuator. The knob may be configured, when moved in a first direction along the longitudinal axis, to move the actuator, which may cause expansion of the expandable member. When moved in a second direction along the longitudinal axis, the knob may compress the expandable member by elongating the actuator along the longitudinal axis. The catheter may include a switch disposed between the knob and the handle so that the movement of the knob in relation to the handle is configured to activate or deactivate a circuit connected to the switch to provide an indication of the knob along the longitudinal axis.

According to some embodiments, the switch can be a proximity sensor switch, an optical sensing switch, a micro lever switch, or combinations thereof. According to some embodiments, the expandable member truncates in length along the longitudinal axis when the knob is moved in the first direction and when the knob is moved in the second direction, the expandable member elongates in length along the longitudinal direction.

According to some embodiments, the switch can be further configured to provide one or more indicates selected from an audible indication, a tactile indication, and a visual indication of the position of the knob in relation to the handle.

According to some embodiments, the catheter can include an irrigation pump that is coupled to the irrigation tubing and in communication with the switch. The irrigation pump can be configured to switch from a first flow rate to a second flow rate in response to an indication from the switch that the knob has been moved in the second direction to contract the expandable member.

According to some embodiments, the console can include a display screen that is in electrical communication with the switch. The display screen can be configured to provide a countdown timer in response to the irrigation pump switching from the first flow rate to the second flow rate. According to some embodiments, the display screen can be further configured to provide a visual indication of a current flow rate of the irrigation pump selected from the first flow rate and the second flow rate. According to some embodiments, the first flow rate can be a high flow rate and the second flow rate can be a low flow rate.

According to some embodiments, the display screen can be further configured to provide a visual indication reminding an operator of the catheter to verify that the catheter is not deflected or torqued before removing the catheter from a patient's vasculature.

According to some embodiments, the expandable member may include one or more electrodes coupled to an outer surface of the expandable member.

According to some embodiments, the catheter can be configured to be applied in a medical procedure selected from electrophysiology, ablation, sinuplasty, surgery, endoscopy, angioplasty, otolaryngology, and neurology.

In another aspect, a method is disclosed. The method may include inserting a distal end of a catheter into a patient organ, wherein the distal end of the catheter may include an expandable balloon. The method may include truncating a length of the expandable balloon by moving a knob, in relation to a handle located approximate a proximal end of the catheter, in a first direction along a longitudinal axis that is defined by the catheter. The method may include providing irrigation fluid from an irrigation pump to an internal volume of the expandable balloon to thereby inflate the expandable balloon, The method may include elongating the length of the expandable balloon by moving the knob, in relation to the handle, in a second direction along the longitudinal axis. The method may include transmitting an electrical signal from a switch positioned on the handle to a device external to the catheter in response to the knob being moved in the second direction. The electrical signal may indicate movement of the knob in the second direction.

According to some embodiments, the device external to the catheter can be an irrigation pump and the method can further include switching the irrigation pump from a first flow rate to a second flow rate in response to the irrigation pump receiving the electrical signal.

According to some embodiments, the device external to the catheter can include a display device. The method can further include displaying, on the display device and in response to the electrical signal, a human readable message instructing a user to reduce a flow rate of an irrigation pump from a first flow rate to a second flow rate in response to the display device receiving the electrical signal.

According to some embodiments, the method can further include providing a first indication to the user that a predetermined amount of time has passed since the irrigation pump has switched to the second flow rate.

According to some embodiments, the first indication can include a countdown presented numerically on the display device.

According to some embodiments, the method can include providing a second indication to remind the user to verify that the distal end of the catheter is aligned with the longitudinal axis defined by the catheter.

According to some embodiments, the second indication can include a message presented on the display device.

According to some embodiments, the method can include causing one or more electrodes to come into contact with a tissue of a patient while the expandable balloon is inflated, where the one or more electrodes are disposed on an outside surface of the expandable balloon. The method can include ablating the tissue with the one or more electrodes. According to some embodiments, the method can include withdrawing the distal end of the catheter from the patient organ when the expandable balloon is deflated.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIGS. 1A and 1B are illustrations of a catheter handle according to aspects of the present invention.

FIG. 1C is an illustration of an interior cross section of a catheter handle according to aspects of the present invention.

FIG. 2A is an illustration of a catheter handle having a knob extended in a first direction, according to aspects of the present invention.

FIG. 2B is an illustration of an expandable balloon in an inflated configuration, according to aspects of the present invention.

FIG. 2C is an illustration of a basket assembly in an expanded configuration, according to aspects of the present invention.

FIG. 3A is an illustration of a catheter handle having a knob extended in a second direction, according to aspects of the present invention.

FIG. 3B is an illustration of an expandable balloon in a deflated configuration, according to aspects of the present invention.

FIG. 3C is an illustration of a basket assembly in a contracted configuration, according to aspects of the present invention.

FIG. 4 is an illustration of a cardiovascular treatment utilizing the catheter handle, according to aspects of the present invention.

FIG. 5 is a detailed view of the basket electrode assembly, according to aspects of the present invention.

FIG. 6 is a detailed view of the expandable balloon, according to aspects of the present invention.

FIG. 7 is a side view of the balloon deployed in a region of a pulmonary vein and its ostium, according to aspects of the present invention.

FIG. 8 is a flowchart of an exemplary method of treating a patient using a catheter handle according to aspects of the present invention.

### DETAILED DESCRIPTION

The following description of certain examples of the invention should not be used to limit the scope of the present invention. The drawings, which are not necessarily to scale, depict selected embodiments and are not intended to limit the scope of the invention. The detailed description illustrates by way of example, not by way of limitation, the principles of the invention. Other examples, features, aspects, embodiments, and advantages of the invention will become apparent to those skilled in the pertinent art from the following description, which is by way of illustration, one of the best modes contemplated for carrying out the invention. As will be realized, the invention is capable of other different or equivalent aspects, all without departing from the invention. Accordingly, the drawings and descriptions should be regarded as illustrative in nature and not restrictive.

Any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those skilled in the pertinent art in view of the teachings herein. Such modifications and variations are intended to be included within the scope of the claims.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±10% of the recited value, e.g. “about 90%” may refer to the range of values from 81% to 99%. In addition, as used herein, the terms “patient,” “host,” “user,” and “subject” refer to any human or animal subject and are not intended to limit the systems or methods to human use, although use of the subject invention in a human patient represents a preferred embodiment.

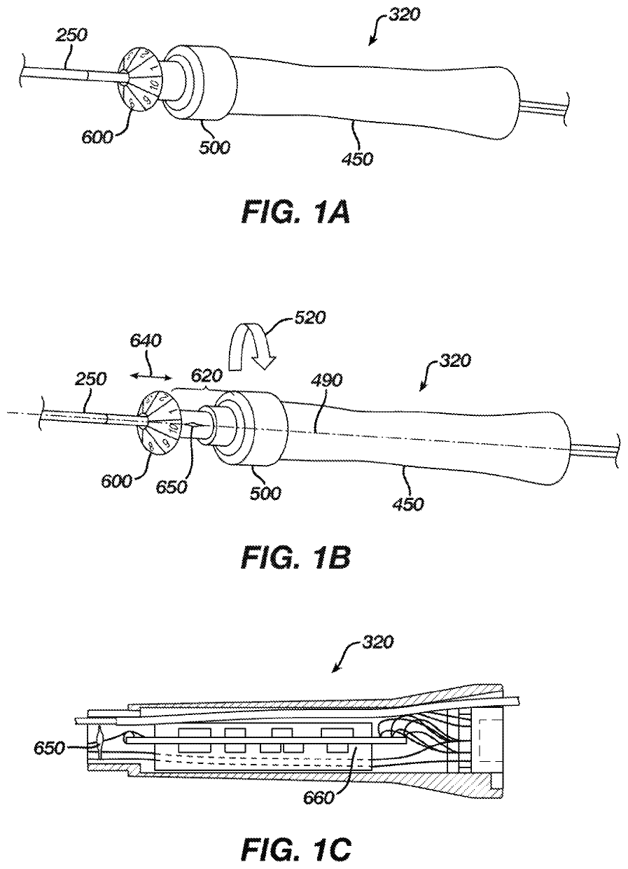

FIGS. 1A and 1B illustrate a catheter handle 320 according to aspects of the present invention. As shown, catheter handle 320 can include an elongated shaft at a distal end of the catheter handle 320. A catheter handle having such features is described in U.S. Pat. No. 10,682,496, the disclosure of which is incorporated herein by reference with a copy provided in the Appendix of priority application U.S. 63/305,757. The catheter handle can include a grip 450 conveniently formed to be handled by a user of catheter handle 320. The catheter handle 320 may be fitted with a first knob 600 on a distal end of the catheter handle 320 and a second knob 500 located proximal to the first knob 600. As shown in FIG. 1B, the catheter handle 320 can define a longitudinal axis 490. The first knob 600 can be configured to move in a direction 640 along the longitudinal axis 490. The adjustment range 620 of first knob 600 is shown in FIG. 1B. As seen in FIG. 1B, a switch 650 may be disposed on catheter handle 320 just proximal of the first knob 600. Additionally, FIG. 1B shows that second knob 500 can be rotated along a second knob rotational adjustment range 520. According to some embodiments, first knob 600 may control the position of actuator 170, which may expand or contract an assembly (e.g., expandable balloon 24 and/or basket assembly 180) located on a distal end of elongated shaft 250. For example, when expandable balloon 24 is elongated by actuator 170 in response to movement of first knob 600 in a distal direction, the expandable balloon is collapsed, forcing inflation fluid through existing holes in the expandable balloon 24. Similarly, expandable balloon 24 can be inflated by the inflation fluid when actuator 170 moves proximally in response to knob 600 being moved in a proximal direction. Basket assembly 180 can be contracted and expanded in a similar manner to expandable balloon 24. According to some embodiments, second knob 500 may control the deflection of an assembly (e.g., expandable balloon 24 and/or basket assembly 180) relative to the longitudinal axis 490. Expandable balloons are further detailed in U.S. Pub. No. 2019/0059818, the disclosure of which is incorporated by reference with a copy provided in the Appendix of priority application U.S. 63/305,757.

According to some embodiments, switch 650 can be configured to provide an indication of a position of first knob 600 in relation to catheter handle 320. The position of first knob 600 may cause an end effector (e.g., an expandable balloon 24, a basket assembly 180, etc.) to either contract or expand, depending on the direction 640 that knob 600 is moved. Rotation 520 of the second knob 500 can be configured to affect an angle of deflection from longitudinal axis 490 of the assembly that is coupled to catheter handle 320.

FIG. 1C is an illustration of an interior cross section of a catheter handle according to aspects of the present invention, which is further described in U.S. Pat. No. 6,024,739, the disclosure of which is incorporated herein by reference with a copy provided in the Appendix of priority application U.S. 63/305,757. As shown in FIG. 1C, switch 650 may be disposed within catheter handle 320 and may be in electrical communication with a circuit 660. Circuit 660 may be configured to activate and deactivate in response to the movement of first knob 600, which provides feedback that can be measured by switch 650 and converted to an electrical signal that is sent to circuit 660. Switch 650 may be of any type known in the art, but preferably a switch selected from an optical sensing switch, a micro lever switch, and/or a proximity sensor switch. According to some embodiments, switch 650 can provide more than non-binary feedback to circuit 660. That is, switch 650 can provide a continuous indication of the position of first knob 600 along catheter handle 320. For example, the switch may output an electrical voltage, for example between approximately 0 volts and approximately 5 volts, that is provided to circuit 660, which is used to determine the position of the knob 600.

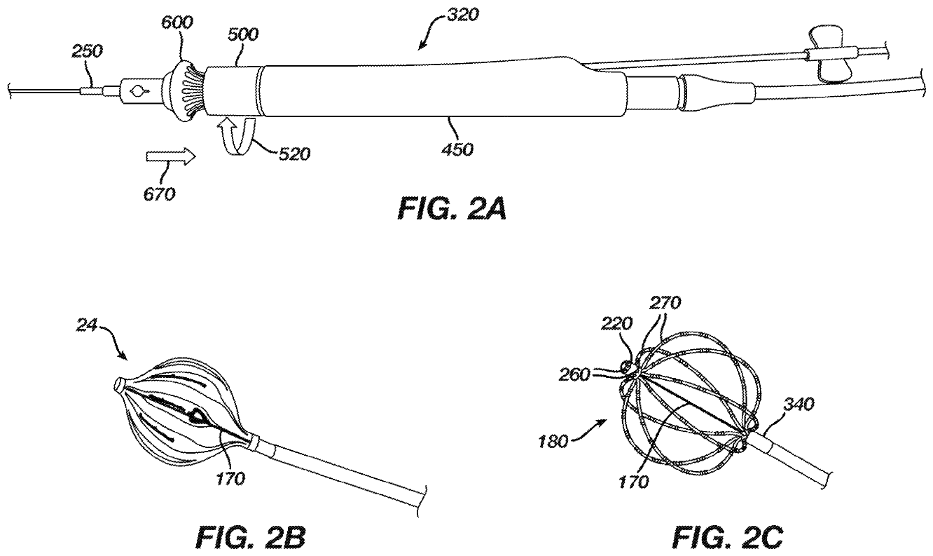

FIG. 2A is an illustration of a catheter handle having a knob extended in a first direction, according to aspects of the present invention. As indicated by arrow 670, first knob 600 is shown moved to the end of adjustment range 620 in a first direction, that is towards proximal end of catheter handle 320. FIG. 2B is an illustration of an expandable balloon 24 in an inflated configuration, according to aspects of the present invention. As shown, an actuator 170 extends through shaft 250 of catheter handle 320 and is attached at a distal end of expandable balloon 24. When first knob 600 is moved in the first direction (e.g., proximally), this provides switch 650 with an indication of the position of first knob 600 in relation to catheter handle 320, which causes an actuator 170 to pull the distal end of expandable balloon 24 proximally towards catheter handle 320. Accordingly, as the actuator 170 compresses expandable balloon 24, the inflation of expandable balloon 24 with an inflation fluid is facilitated.

FIG. 2C is an illustration of a basket assembly 180 in an expanded configuration, according to aspects of the present invention. Basket assembly is further described in U.S. Pat. No. 9,314,208, which is incorporated by reference herein with a copy in the Appendix of priority application U.S. 63/305,757. As shown, basket assembly 180 includes outer tubing 340 connecting basket assembly 180 to elongated shaft of catheter handle 320. Actuator 370 is disposed within an inner lumen of outer tubing 340 and extends through elongated shaft 250 and attaches to a distal end of the basket assembly 180 at a distal tip 220. Disposed on distal tip 220 can be a microelectrode array 260, which may be utilized for medical procedures (e.g., ablation).

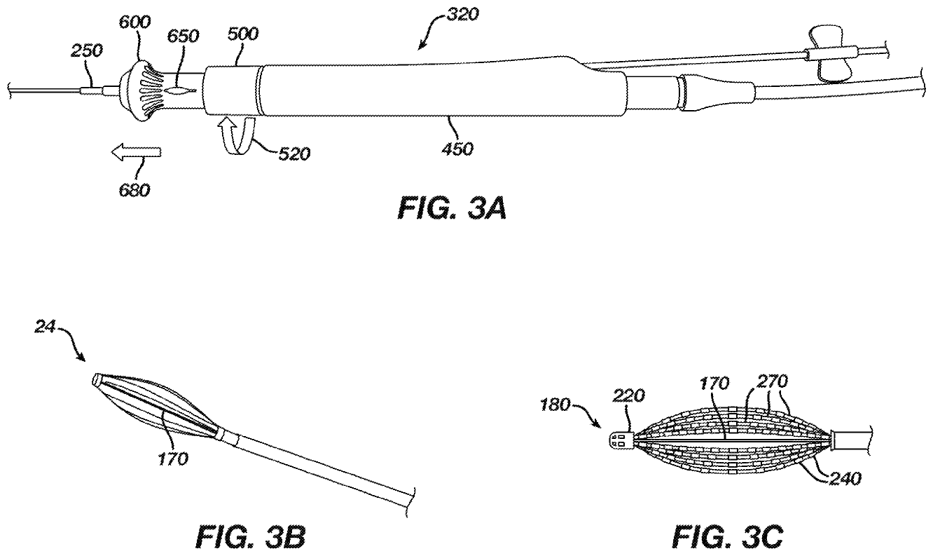

FIG. 3A is an illustration of a catheter handle having a knob extended in a second direction, according to aspects of the present invention. As indicated by arrow 680, first knob 600 is shown moved to the end of adjustment range 620 in a second direction, that is towards distal end of catheter handle 320. FIG. 3B is an illustration of an expandable balloon 24 in a deflated configuration, according to aspects of the present invention. As shown, an actuator 170 extends through shaft 250 of catheter handle 320 and is attached at a distal end of expandable balloon 24. When first knob 600 is moved in the second direction (e.g., distally), this provides switch 650 with an indication of the position of first knob 600 in relation to catheter handle 320, which causes actuator 170 to push the distal end of expandable balloon 24 distally away from catheter handle 320. Accordingly, as the catheter elongates expandable balloon 24, the expandable balloon 24 is collapsed, pushing the inflation fluid out of expandable balloon 24, thereby facilitating deflation of the expandable balloon 24.

FIG. 3C is an illustration of a basket assembly in a contracted configuration, according to aspects of the present invention and as further shown in U.S. Pat. No. 9,314,208, which is incorporated by reference herein with a copy in the Appendix of priority application U.S. 63/305,757. As shown, basket assembly 180 is in a collapsed state caused by actuator 170 pushing the distal end of basket assembly 180 distally away from catheter handle 320.

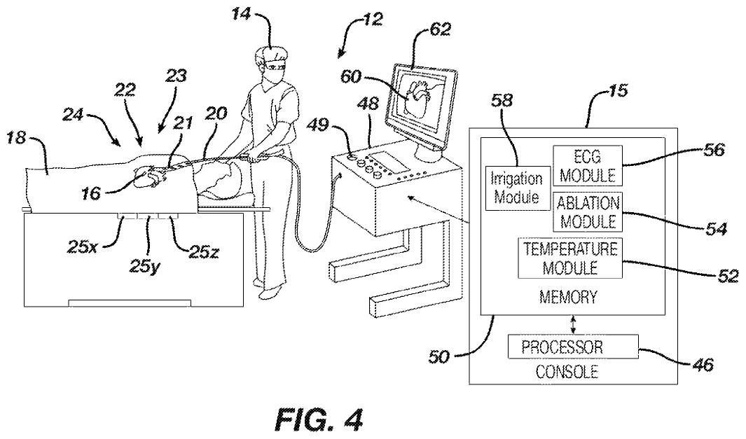

FIG. 4 is an illustration of a cardiovascular treatment utilizing the catheter handle, according to aspects of the present invention. Cardiovascular treatment is further shown and described in U.S. Pat. Pub. No. 2018/0161093, which is incorporated by reference herein with a copy in the Appendix of priority application U.S. 63/305,757. As shown in FIG. 4, apparatus 12 is controlled by a system process 46, which is located in an operating console 15 of the apparatus. Console 15 includes controls 49 which are used by professional 14 to communicate with the processor. During the procedure, the processor 46 typically tracks a location and an orientation of the distal end 22 of the probe 20, using any method known in the art. For example, processor 46 may use a magnetic traffic method, wherein magnetic transmitters 25x, 25y, and 25z external to the patient 18 generate signals in coils positioned in the distal end of the probe 20. The CARTO® available from Biosense Webster, Inc. of Diamond Bar, Calif., uses such a tracking method.

The software for the processor 46 may be downloaded to the processor in electronic form, over a network, for example. Alternatively or additionally, the software may be provided on non-transitory tangible media, such as optical, magnetic, or electronic storage media. The tracking of the distal end 22 is typically displayed on a three-dimensional representation 60 of the heart of the patient 18 on a screen 62.

In order to operate apparatus 12, the processor 46 communicates with a memory 50, which has a number of modules used by the processor to operate the apparatus. Thus, the memory 50 can include a temperature module 52, an ablation module 54, an electrocardiograph (ECG) module 56, and an irrigation module 58 for controlling irrigation provided for the distal end 22. The memory 50 typically includes other modules, such as a force module for measuring the force on the distal end 22, and a tracking module for operating the tracking method used by the processor 46. For simplicity, such other modules are not illustrated in FIG. 4. The modules may include hardware as well as software elements.

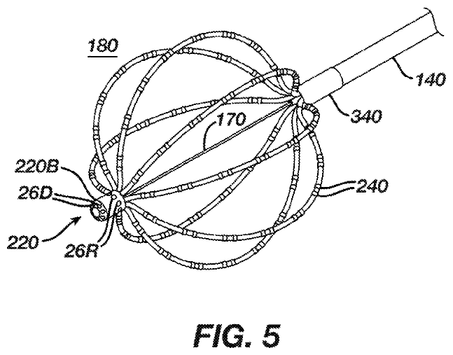

FIG. 5 is a detailed view of the basket electrode assembly, according to aspects of the present invention, and as further detailed in U.S. Pat. No. 9,314,208, which is incorporated by reference herein with a copy in the Appendix of priority application U.S. 63/305,757. The basket assembly 180 may be mounted to the distal end of elongated shaft 250 at a deflection section 140. Deflection section 140 is configured to allow the basket assembly 180 to deflect from the longitudinal axis 490 in response to axial rotation of the second knob 500. Proximal of deflection section 140 may be outer tubing 340 that extends a short distance from the distal end of deflection section 140. The outer tubing may be made of any suitable material, for example, PEEK (polyetheretherketone). Inside of outer tubing may be a disc with a plurality of through-holes to receive and position actuator 170 and cabling 210 that connects to ring electrodes 240. Ring electrodes 240 may be configured as monopolar or bipolar, as known in the art. Distal ends of cabling 210 can converge around the distal end of actuator 170 in the distal tip 22 of basket assembly 180. The distal tip 22 has a generally solid, elongated, nonmetallic, electrically-insulating substrate body 25 with a generally cylindrical shape (with a two-dimensional curvature in the X/Y direction and a linear length in the Z direction), and a domed distal end (with a three-dimensional curvature in the X/Y/Z direction). Radial microelectrodes 26R are located on radial outer surface of the body 25. Distal microelectrodes 26D are located on distal outer surface of the body 25. Each radial microelectrode 26R and distal microelectrode 26D are electrically connected with a respective lead wire extending through elongated body 250 of the basket assembly 180.

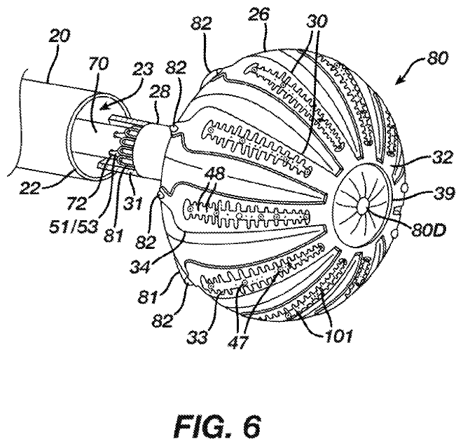

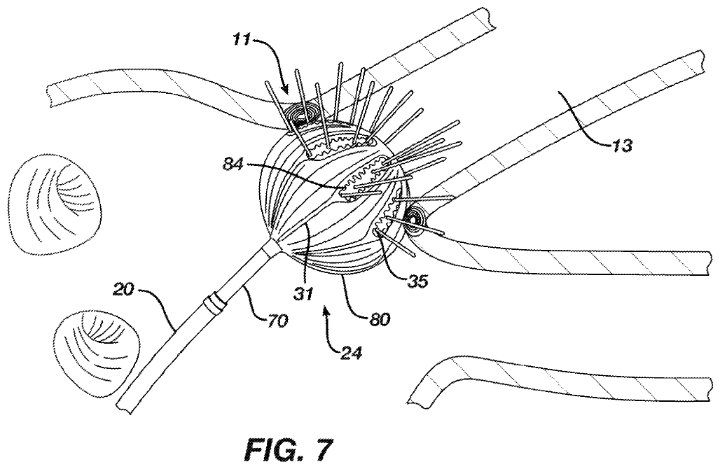

FIG. 6 is a detailed view of the expandable balloon, according to aspects of the present invention. The expandable balloon is further described in U.S. Pat. Pub. No. 2018/0161093, which is incorporated by reference herein with a copy in the Appendix of priority application U.S. 63/305,757. As shown in FIG. 6, expandable balloon 24 is in an inflated configuration. In a disclosed embodiment, the expandable balloon 24 may be used to ablate an ostium 11 of a lumen, such as a pulmonary vein 13, as shown in FIG. 4. The expandable balloon 24 may extend from a distal end of elongated shaft 250. In some embodiments, the inflation balloon 24 has an exterior wall or membrane 26 of a bio-compatible material, for example, formed from a plastic such as polyethylene terephthalate (PET), polyurethane, or PEBAX®. The elongated shaft 250 may define a longitudinal axis. The expandable balloon can be deployed in a collapsed (e.g., deflated) configuration through lumen 23, and may be inflated after existing from the distal end 22 of elongated shaft 250. The expandable balloon 24 may be inflated and deflated by inflation fluid, such as a saline solution through the catheter shaft. The membrane 26 of the expandable balloon 24 is formed with irrigation pores or apertures 27 through which the fluid can exit from the interior of the expandable balloon 24 to outside the balloon for cooling the tissue ablation site. While FIG. 7 shows fluid existing the expandable balloon 24 as jet streams, it is understood that the fluid may exist the balloon with any desired flow rate and/or pressure, including a rate where the fluid is seeping out of the apertures 27.

The membrane 26 can support and carry a combined electrode and temperature sensing member which is constructed as a multi-layer flexible circuit electrode assembly 84. The “flex circuit electrode assembly” 84 may have many different geometric configurations. In the illustrated embodiment, the flex circuit electrode assembly 84 has a plurality of radiating leaves or strips 30. The leaves 30 are evenly distributed about the distal end 80D of expandable balloon 24. Each leaf can have a wider proximal portion that gradually tapers to a narrower distal portion.

Each leaf 30 may have a proximal tail 31 and may be connected at its distal end to a hub 32 with a central opening 39 that is concentric with the distal end 80D of the expandable balloon 24. The proximal tail 31 can be tucked under and fastened to the elongated tube 250 by a proximal ring 28 mounted on internal shaft 70. One or more contact electrodes 33 on each leaf can come into galvanic contact with the ostium 11 during an ablation procedure, during which electrical current flows from the contact electrodes 33 to the ostium 11, as shown in FIG. 7. Lead wires 51, 53 may connect contact electrode 33 to console 15 and circuit 660. One or more through-holes 72 may be provided situated at different radial locations around the internal shaft 70. Lead wires 51 and 53 may enter the lumen of internal shaft 70 through the one or more through-holes 72.

Formed within the contact electrode 33 can be one or more conductive blind vias 48 which are conductive or metallic formations or substances that extend through through-holes (not shown) in substrate 34 and are configured as electrical conduits connecting the contact electrode 33 and a wiring electrode that may be sandwiched between the substrate 34 and the balloon membrane 26. It is understood that “conductive” is used herein interchangeably with “metallic” in all relevant instances. Formed within the contact electrode 33 can be one or more exclusion zones 47, which each can surround an irrigation aperture (not shown) formed in substrate 34 which is in communication with a corresponding irrigation aperture formed in the balloon membrane 26. According to some embodiments, expandable balloon 24 may include microelectrodes 110, which can be physically and electrically isolated from a partially or fully surrounding contact electrode. The microelectrodes can be configured for impedance, electrical signals, and/or temperature sensing independently of the other electrodes (e.g., electrodes 33).

As shown in FIG. 6, the expandable balloon 24 includes a plurality of elongated longitudinal supports or “spines” 81 extending radially from a proximal or distal end of the expandable balloon 24 to a location on the outer surface of the balloon membrane 26 proximal to the distal end. Or distal to the proximal end. That is, ends of the spines 81 fall around an equatorial portion of the expandable balloon 24. The support spines 81 are made of a suitable material with shape-memory, for example, nitinol. The spines may have any suitable cross sectional shape, e.g., rectangular or circular, and can be hollow and preshaped with a curvature to ensure that the expandable balloon 24 assumes a generally spherical configuration when deployed from the distal end of the elongated shaft 250, especially when inflated with inflation fluid. In some embodiments, each spine 81 is covered by a cover 82 configured, e.g., as a strip or a sleeve, that is affixed to an outer surface of the balloon membrane 26 and provides an interior passage through which the spine 81 extends.

FIG. 7 is a side view of the balloon deployed in a region of a pulmonary vein and its ostium, according to aspects of the present invention. The expandable balloon is further described in U.S. Pat. Pub. No. 2018/0161093, which is incorporated by reference herein with a copy in the Appendix of priority application U.S. 63/305,757. As shown in FIG. 7, expandable balloon 24 may be used to ablate an ostium 11 of a lumen, such as pulmonary vein 13. The expandable balloon 24 extends at the distal end of the elongated shaft 250. One or more contact electrodes 33 on each leaf can come into galvanic contact with the ostium, and the contact electrodes 33 can deliver RF energy to the ostium during ablation, or may be connected to a thermocouple junction for temperature/electropotential sensing of the ostium.

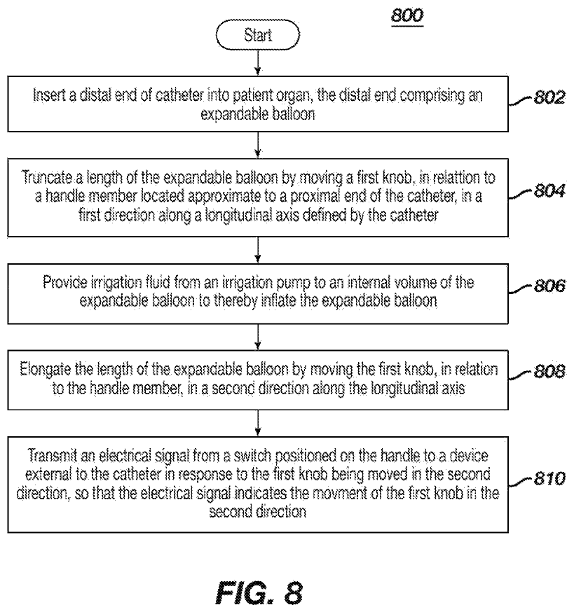

FIG. 8 is a flowchart of an exemplary method 800 of treating a patient using a catheter handle according to aspects of the present invention. In block 802, the method may include inserting a distal end of a catheter into a patient organ. The distal end of the catheter can include an expandable balloon (e.g., expandable balloon 24). In block 804, the method may include truncating a length of the expandable balloon by moving a first knob in relation to a handle member of the catheter. The first knob may be moved in a first direction (e.g., proximally) along a longitudinal axis that is defined by the catheter (e.g., longitudinal axis 490). In block 806, the method may include providing irrigation fluid from an irrigation pump to an internal volume of the expandable balloon. The irrigation fluid may inflate the expandable balloon. In block 808, the method may include elongating the length of the expandable balloon by moving the first knob in relation to the handle member in a second direction (e.g., distally) along the longitudinal axis (e.g., longitudinal axis 490). In block 810, the method may include transmitting an electrical signal from a switch positioned on the handle to a device that is external to the catheter. The electrical signal may be transmitted in response to the first knob being moved in the second direction so that the electrical signal indicates the movement of the first knob in the second (e.g., distal) direction.

According to some embodiments, the external device may include an irrigation pump, and the method may further include switching the irrigation pump from a first flow rate (e.g., a high flow rate) to a second flow rate (e.g., a low flow rate) in response to the irrigation pump receiving the electrical signal.

According to some embodiments, the external device may include a display device, and the method may further include displaying on the display device, and in response to the electrical signal, a human readable message instructing a user to reduce a flow rate of an irrigation pump from a first flow rate to a second flow rate in response to the display device receiving the electrical signal.

According to some embodiments, the method may additionally include providing a first indication to a user that a predetermined amount of time has passed since the irrigation pump switched from the first flow rate to the second flow rate. According to some embodiments, the first indication can include a countdown presented numerically on the display device.

According to some embodiments, the method may include providing a second indication to the user (e.g., professional 14) reminding the user to verify that the distal end of the catheter is aligned with the longitudinal axis (e.g. longitudinal axis 490) defined by the catheter. For example, the user may turn second knob 500 in order to adjust the deflection angle of the expandable balloon 24 with respect to the longitudinal axis 490 before withdrawing the device from the patient organ.

According to some embodiments, the method may include causing one or more electrodes disposed on an outside surface of the expandable balloon to come into contact with a tissue of a patient, and ablating the tissue with the one or more electrodes. According to some embodiments, the method may include withdrawing the distal end of the catheter from the patient organ.

The disclosed technology described herein can be further understood according to the following clauses:

Clause 1. A medical instrument, comprising: an expandable balloon disposed approximate a distal end of the medical instrument, the expandable balloon comprising an internal volume configured to receive inflation fluid; a handle disposed approximate a proximal end of the medical instrument; a knob extending from the handle which is movable in relation to the handle along a longitudinal axis of the medical instrument such that when the handle moves in a first direction along the longitudinal axis the expandable balloon truncates in length along the longitudinal axis to allow inflation of the expandable balloon and such that when the handle moves in a second direction along the longitudinal axis the expandable balloon elongates to facilitate deflation of the expandable balloon; and a switch disposed between the handle and the knob so that movement of the knob relative to the handle activate and deactivate a circuit connected to the switch and provide an indication of a position of the knob, in relation to the handle, along the longitudinal axis.

Clause 2. The medical instrument of clause 1, wherein the switch is selected from a group of switches comprising at least one proximity sensor switch, optical sensing switch, micro lever switch and combinations thereof.

Clause 3 The medical instrument of clauses 1 or 2, wherein the switch is further configured to provide one or more indications selected from an audible indication, a tactile indication, and a visual indication of the position of the knob in relation to the handle.

Clause 4. The medical instrument of any of clauses 1 to 3, wherein the switch is further configured to provide a signal to a console in electrical communication with the switch that indicates the position of the knob in relation to the handle.

Clause 5. The medical instrument of clause 4, further comprising: an irrigation pump in electrical communication with the switch, the irrigation pump configured to switch from a first flow rate to a second flow rate in response to an indication from the switch that the knob has been moved in the second direction to deflate the expandable balloon, the irrigation pump configured to provide the inflation fluid to the expandable balloon in response to the knob being moved in the first direction along the longitudinal axis.

Clause 6. The medical instrument of clause 5, wherein the console further comprises a display screen, in electrical communication with the switch, that is configured to provide a countdown timer in response to the irrigation pump switching from the first flow rate to the second flow rate.

Clause 7. The medical instrument of clause 6, wherein the display screen is further configured to provide a visual indication of a current flow rate of the irrigation pump selected from the first flow rate and the second flow rate.

Clause 8. The medical instrument of clause 7, wherein the first flow rate is a high flow rate and the second flow rate is a low flow rate.

Clause 9. The medical instrument of any of clause 6 to 8, wherein the display screen is further configured to provide a visual indication reminding an operator of the medical instrument to verify that the medical instrument is not deflected or torqued before removing the medical instrument from a patient's vasculature.

Clause 10. The medical instrument of any of clauses 1 to 9, wherein the expandable balloon comprises one or more electrodes coupled to an outer surface of the expandable balloon.

Clause 11. The medical instrument of any of clauses 1 to 10, wherein the medical instrument is configured to be applied in a medical procedure selected from a list consisting of electrophysiology, ablation, sinuplasty, surgery, endoscopy, angioplasty, otolaryngology and neurology.

Clause 12. A catheter, comprising: an elongated catheter shaft having a first lumen and defining a longitudinal axis, the elongated catheter shaft having a proximal end and a distal end, the proximal end defining a handle: an expandable member distal of the elongated catheter shaft, the expandable member having a distal end and a proximal end and an actuator disposed along the longitudinal axis and connected to the distal end of the expandable member for movement relative thereto; an irrigation tubing extending through the first lumen of the elongated catheter shaft, the irrigation tubing having a second lumen, the irrigation tubing having a distal end terminating at the proximal end of the expandable member; a knob, which is fitted on the handle, and which is movable along the longitudinal axis to control the actuator, wherein the knob is configured, when moved in a first direction along the longitudinal axis, to move the actuator causing expansion of the expandable member and when moved in a second direction along the longitudinal axis, to compress the expandable member by elongating the actuator along the longitudinal axis; and a switch disposed between the knob and the handle so that movement of the knob in relation to the handle activate or deactivate a circuit connected to the switch and provide an indication of a position of the knob along the longitudinal axis.

Clause 13. The catheter of clause 12, wherein the switch is selected from a group of switches comprising at least one proximity sensor switch, optical sensing switch, micro lever switch and combinations thereof.

Clause 14. The catheter of clause 12, wherein when the knob is moved in the first direction, the expandable member truncates in length along the longitudinal axis, and wherein when the knob is moved in the second direction, the expandable member elongates in length along the longitudinal axis.

Clause 15. The catheter of any of clauses 12 to 14, wherein the switch is further configured to provide one or more indications selected from an audible indication, a tactile indication, and a visual indication of the position of the knob in relation to the handle.

Clause 16. The catheter of any of clauses 12 to 15, wherein the switch is further configured to provide a signal to a console in electrical communication with the switch that indicates the position of the knob in relation to the handle.

Clause 17. The catheter of clause 16, further comprising: an irrigation pump coupled to the irrigation tubing and in communication with the switch, the irrigation pump configured to switch from a first flow rate to a second flow rate in response to an indication from the switch that the knob has been moved in the second direction to contract the expandable member.

Clause 18. The catheter of clause 17, wherein the console further comprises a display screen, in electrical communication with the switch, that is configured to provide a countdown timer in response to the irrigation pump switching from the first flow rate to the second flow rate.

Clause 19. The catheter of clause 18, wherein the display screen is further configured to provide a visual indication of a current flow rate of the irrigation pump selected from the first flow rate and the second flow rate.

Clause 20. The catheter of any of clause 17 to 19, wherein the first flow rate is a high flow rate and the second flow rate is a low flow rate.

Clause 21. The catheter of any of clauses 18 to 20, wherein the display screen is further configured to provide a visual indication reminding an operator of the catheter to verify that the catheter is not deflected or torqued before removing the catheter from a patient's vasculature.

Clause 22. The catheter of any of clauses 12 to 21, wherein the expandable member comprises one or more electrodes coupled to an outer surface of the expandable member.

Clause 23. The catheter of any of clauses 12 to 22, wherein the catheter is configured to be applied in a medical procedure selected from a list consisting of electrophysiology, ablation, sinuplasty, surgery, endoscopy, angioplasty, otolaryngology and neurology.

Clause 24. A method, comprising: inserting a distal end of a catheter into a patient organ, the distal end comprising an expandable balloon; truncating a length of the expandable balloon by moving a knob, in relation to a handle located approximate a proximal end of the catheter, in a first direction along a longitudinal axis defined by the catheter; providing irrigation fluid from an irrigation pump to an internal volume of the expandable balloon to thereby inflate the expandable balloon; elongating the length of the expandable balloon by moving the knob, in relation to the handle, in a second direction along the longitudinal axis; and transmitting an electrical signal from a switch positioned on the handle to a device external to the catheter in response to the knob being moved in the second direction, so that the electrical signal indicates movement of the knob in the second direction.

Clause 25. The method of clause 24, wherein the device external to the catheter comprises an irrigation pump, the method further comprising: switching the irrigation pump from a first flow rate to a second flow rate in response to the irrigation pump receiving the electrical signal.

Clause 26. The method of clause 24, wherein the device external to the catheter comprises a display device, the method further comprising: displaying, on the display device, in response to the electrical signal, a human readable message instructing a user to reduce a flow rate of an irrigation pump from a first flow rate to a second flow rate in response to the display device receiving the electrical signal.

Clause 27. The method of claim 26, further comprising: providing a first indication to the user that a predetermined amount of time has passed since the irrigation pump has switched to the second flow rate.

Clause 28. The method of claim 27, wherein the first indication comprises a countdown presented numerically on the display device.

Clause 29. The method of claim 27 or 28, further comprising: providing a second indication to remind the user to verify that the distal end of the catheter is aligned with the longitudinal axis defined by the catheter.

Clause 30. The method of claim 29, wherein the second indication comprises a message presented on the display device.

Clause 31. The method of any of claims 24 to 30, further comprising: while the expandable balloon is inflated, causing one or more electrodes disposed on an outside surface of the expandable balloon to come into contact with a tissue of a patient; and ablating the tissue with the one or more electrodes.

Clause 32. The method of any of claims 24 to 31, further comprising: when the expandable balloon is deflated, withdrawing the distal end of the catheter from the patient organ.

What is claimed is:

1. A medical instrument, comprising:
an expandable balloon disposed approximate a distal end of the medical instrument, the expandable balloon comprising an internal volume configured to receive inflation fluid;
a handle disposed approximate a proximal end of the medical instrument;
a knob extending from the handle which is movable in relation to the handle along a longitudinal axis of the medical instrument such that when the handle moves in a first direction along the longitudinal axis the expandable balloon truncates in length along the longitudinal axis to allow inflation of the expandable balloon and such that when the handle moves in a second direction along the longitudinal axis the expandable balloon elongates to facilitate deflation of the expandable balloon; and
a switch disposed between the handle and the knob so that movement of the knob relative to the handle activate and deactivate a circuit connected to the switch and provide an indication of a position of the knob, in relation to the handle, along the longitudinal axis.

2. The medical instrument of claim 1, wherein the switch is selected from a group of switches comprising at least one proximity sensor switch, optical sensing switch, micro lever switch and combinations thereof.

3. The medical instrument of claim 1, wherein the switch is further configured to provide one or more indications selected from an audible indication, a tactile indication, and a visual indication of the position of the knob in relation to the handle.

4. The medical instrument of claim 1, wherein the switch is further configured to provide a signal to a console in electrical communication with the switch that indicates the position of the knob in relation to the handle.

5. The medical instrument of claim 4, further comprising:
an irrigation pump in electrical communication with the switch, the irrigation pump configured to switch from a first flow rate to a second flow rate in response to an indication from the switch that the knob has been moved in the second direction to deflate the expandable balloon, the irrigation pump configured to provide the inflation fluid to the expandable balloon in response to the knob being moved in the first direction along the longitudinal axis.

6. The medical instrument of claim 5, wherein the console further comprises a display screen, in electrical communication with the switch, that is configured to provide a countdown timer in response to the irrigation pump switching from the first flow rate to the second flow rate.

7. The medical instrument of claim 6, wherein the display screen is further configured to provide a visual indication of a current flow rate of the irrigation pump selected from the first flow rate and the second flow rate.

8. The medical instrument of claim 7, wherein the first flow rate is a high flow rate and the second flow rate is a low flow rate.

9. The medical instrument of claim 8, wherein the display screen is further configured to provide a visual indication reminding an operator of the medical instrument to verify that the medical instrument is not deflected or torqued before removing the medical instrument from a patient's vasculature.

10. The medical instrument of claim 1, wherein the expandable balloon comprises one or more electrodes coupled to an outer surface of the expandable balloon.

11. The medical instrument of claim 1, wherein the medical instrument is configured to be applied in a medical procedure selected from a list consisting of electrophysiology, ablation, sinuplasty, surgery, endoscopy, angioplasty, otolaryngology and neurology.

12. A catheter, comprising:
an elongated catheter shaft having a first lumen and defining a longitudinal axis, the elongated catheter shaft having a proximal end and a distal end, the proximal end defining a handle;
an expandable member distal of the elongated catheter shaft, the expandable member having a distal end and a proximal end and an actuator disposed along the longitudinal axis and connected to the distal end of the expandable member for movement relative thereto;
an irrigation tubing extending through the first lumen of the elongated catheter shaft, the irrigation tubing having a second lumen, the irrigation tubing having a distal end terminating at the proximal end of the expandable member;
a knob, which is fitted on the handle, and which is movable along the longitudinal axis to control the actuator, wherein the knob is configured, when moved in a first direction along the longitudinal axis, to move the actuator causing expansion of the expandable member and when moved in a second direction along the longitudinal axis, to compress the expandable member by elongating the actuator along the longitudinal axis; and
a switch disposed between the knob and the handle so that movement of the knob in relation to the handle activate or deactivate a circuit connected to the switch and provide an indication of a position of the knob along the longitudinal axis.

13. The catheter of claim 12, wherein the switch is selected from a group of switches comprising at least one proximity sensor switch, optical sensing switch, micro lever switch and combinations thereof.

14. The catheter of claim 12,
wherein when the knob is moved in the first direction, the expandable member truncates in length along the longitudinal axis, and
wherein when the knob is moved in the second direction, the expandable member elongates in length along the longitudinal axis.

15. The catheter of claim 12, wherein the switch is further configured to provide one or more indications selected from an audible indication, a tactile indication, and a visual indication of the position of the knob in relation to the handle.

16. The catheter of any of claim 12, wherein the switch is further configured to provide a signal to a console in electrical communication with the switch that indicates the position of the knob in relation to the handle.

17. The catheter of claim 16, further comprising:
an irrigation pump coupled to the irrigation tubing and in communication with the switch, the irrigation pump configured to switch from a first flow rate to a second flow rate in response to an indication from the switch that the knob has been moved in the second direction to contract the expandable member.

18. The catheter of claim 17, wherein the console further comprises a display screen, in electrical communication with the switch, that is configured to provide a countdown timer in response to the irrigation pump switching from the first flow rate to the second flow rate.

19. The catheter of claim 18, wherein the display screen is further configured to provide a visual indication of a current flow rate of the irrigation pump selected from the first flow rate and the second flow rate.

20. The catheter of claim 17, wherein the first flow rate is a high flow rate and the second flow rate is a low flow rate.
